(19) United States
(12) Patent Application Publication
(10) Pub. No.: US 2010/0191075 A1
(43) Pub. Date: Jul. 29, 2010

(54) Progressively Personalized Decision-Support Menu for Controlling Diabetes

(76) Inventor: Kimon Angelides, Houston, TX (US)

Correspondence Address: ERIC P. MIRABEL, 3783 DARCUS, HOUSTON, TX, 77005, US

(21) Appl. No.: 12/693,849

(22) Filed: Jan. 26, 2010

Related U.S. Application Data
(60) Provisional application No. 61/147,157, filed on Jan. 26, 2009.

(51) Int. Cl. A61B 5/00; G06Q 50/00; G06F 3/048; G06F 15/16; G10L 21/00

(52) U.S. Cl. 600/301; 705/2; 715/811; 709/219; 704/270

(57) ABSTRACT

A method for more effective control and treatment of diabetes is described, based on providing patients readily accessible and real-time analysis and recommendations based on their individual blood glucose levels and other biometric parameters, particularly blood chemistry, diet and exercise. The system includes the ability to track individual blood glucose and wellness responses to changes in insulin dosage and frequency, diet and exercise levels, and to personalize responses and recommendations, all in real time, and on a portable device which can be carried by the patient.

### RELATED APPLICATIONS

This application claims priority to U.S. Provisional Application No. 61/147157, filed Jan. 26, 2009.

### FIELD

The invention relates to transmitting personalized diabetes-related treatment options to patients remotely.

### BACKGROUND

As America's fifth-deadliest disease, there are about 20.8 million American diabetics, diabetes mellitus places a particularly high expense burden on the public healthcare system. As many as 6.2 million Americans are not even aware that they have the disease, and an additional 54 million Americans have pre-diabetes. If the present trends continues, 1 in 3 Americans, including as many as 1 in 2 minorities born in 2000 will develop diabetes in their lifetime.

Diabetes is a group of chronic metabolic diseases marked by high levels of blood glucose resulting from defects in insulin production, insulin action, or both. While diabetes can lead to serious complications and premature death, effective treatment requires the diabetic patient to take steps to control the disease and lower the risk of complications.

About 5-10% of diabetics have Type 1 while 90-95% have Type 2 diabetes. Type 1 is an autoimmune disease while Type 2 results from insulin resistance or inadequate insulin production. Type 1 has clear genetic markers while Type 2 is genetically heterogenous and therefore has a broader and less certain origin. About 80% of Type 2 diabetics are overweight.

Since 1987, the death rate due to diabetes has increased by 45 percent, while the death rates due to heart disease, stroke, and cancer have declined, emphasizing both the failures of the current treatment approaches as well as the rapid growth of this disease.

Uncontrolled diabetes leads to chronic end-stage organ disease and in the United States is a leading cause of end-stage renal disease, blindness, non-traumatic amputation, and cardiovascular disease. It is also associated with complications such as:
Heart Disease and Stroke (#1 cause of death for diabetics and 2-4 time higher than the general population)
High Blood Pressure (3 in 4 diabetics)
Nervous System Damage (can lead to amputations and carpel tunnel syndrome)
Pregnancy Complications (including gestational diabetes)
Sexual Dysfunction (double the incidence of erectile dysfunction)
Periodontal Disease

In the USA, as many as 87.7% of people aged 65 and over have diabetes, a fact that complicates their total health picture and often accelerates chronic end-stage disease, adding an enormous strain to the healthcare system. Prevalence is highest among minorities and increases in all groups with age and obesity. In addition, there are correlations of higher diabetes incidence with smokers, and Alzheimer's patients.

Poor control of blood-glucose in diabetes dramatically increases the risk of heart disease stroke, amputations, blindness, renal disease and failure, impotence, and many other diseases—better control of blood-glucose levels greatly mitigates these complications. Coupled with proper education, nutrition, maintenance of stable blood-glucose levels, and regular exercise, many Type 1 and 2 diabetics can minimize the effects of the disease.

With the growing problem of diabetes in developed and developing countries, comes a growing need for convenient blood glucose monitoring, and convenient methods for analysis and treatment based on the monitoring. Diabetes patients need to monitor their blood glucose multiple times a day and record this information, which is analyzed, along with other parameters such as quantity of exercise and their diet, and then used to adjust the dosage of insulin and/or other therapeutic agent and the recommended quantity of exercise; and a diet. The analysis and adjustment is done with a complex algorithm/decision support work flow, which changes as research advances, new therapies enter the market, and the individual patient experience and responses are collected. What is equally important is that the information that is provided back to the patient be personalized and tailored to the individual's needs and environment.

There are a number of systems for automatically recording blood glucose and other parameters in a convenient form, including wireless systems where the data is transmitted to a database from the patient. However, no system provides a progressively updated and individually-selected (from a number of menus) decision support work flow menu to the patient based on analysis of blood glucose and other personal data that is patient-submitted, and that includes the ability to track and respond to individual differences and responses to insulin dosage and frequency, diet and exercise level and other biometric data, all analyzed to provide real-time personalized and invidualized instructions to the patient

### SUMMARY

A method for more effective control and treatment of diabetes is described, based on providing patients readily accessible and real-time analysis and recommendations based on their individual blood glucose levels and other biometric parameters, including particularly diet and exercise. The system includes the ability to track individual blood glucose and wellness responses to changes in insulin dosage and frequency, diet and exercise levels, and other biometric data, and to personalize the analysis and menu responses, all in real time.

A convenient, portable, and user-friendly system is platformed on a cellular phone—where a Bluetooth or other wireless compatible device functions as a blood glucose analyzer, and a pedometer or other measuring device that collects biometric patient data, which are analyzed and, based on the analysis, a set of decision support menus are displayed for the patient. The Bluetooth or other wireless compatible device (e.g., the Cellular GPRS-communication linked glucometer-pedometer, described in U.S. application Ser. No. 12/426,984, filed Apr. 21, 2009; incorporated by reference) can be used for doing on board calculations and menu selection, and then transmitting this information directly to a database, which can be retrieved by a cell phone, from a personal computer, or from any internet connection. Such a wireless compatible device may be even more portable than a cellular phone, and permits the user to wear it as a necklace or otherwise readily transport it with them. A related alternative is to have a cellular phone equipped with a glucometer embedded in the device (also as described in Ser. No. 12/426,984), where the phone itself can receive and display the decision support menus. In either case, the user can enter data, and receive analysis and advice anywhere there is cellular phone reception. Users are provided individualized menus, depending on their present or past experiences, condition, and data input.

### BRIEF DESCRIPTION OF THE DRAWINGS

FIG. 1 is a block diagram showing an embodiment of a wireless patient support system, including a glucometer and pedometer.

### DETAILED DESCRIPTION

In a preferred embodiment, blood glucose level, diet or other biometric data entered by the patient, or are automatically tracked by a pedometer or by a built-in glucometer, or by any other biometric monitoring device for parameters including blood pressure, blood oxygenation levels, pulse rate, or blood chemistry including cholesterol and ketone level. This data is transmitted, at intervals, to a server, wherein, based on said blood glucose level, pedometer readings, and possibly additional biometric parameters, a particular work flow-decision support menu of possible treatments is selected from a set of such menus and transmitted from the server to the individual. The patient goes through the menu, and is directed to appropriate treatments based on the blood glucose level as determined by the glucometer, and additionally, based on answers to a series of questions, including, e.g., “When and what did you last eat”? “How do you feel”? Based on the answers and the blood glucose and the glucose levels and other biometric data, certain options are automatically selected for the patient, and/or he is addressed with further questions. Instead of conventional strip-test glucometers, Page: 3 other types of glucometers can be used, including blood glucose monitors where a sensor is implanted under the skin that continuously measures and provides interstitial fluid blood glucose.

The system has the ability to track the response (through blood glucose levels and possibly other biometric parameters) to variations in insulin, diet (input by the patient) and exercise (from the pedometer). The system also has the ability to adjust its subsequent instructions and menus in light of the individual's responses. Through this continuing adjustment, the instructions are progressively personalized in real-time to the user, and thus are a personalized user-specific response.

Alternatively, at any point where there is a perceived need for immediate expert consultation or advice, the system would automatically alert health care professionals to intervene and provide it (which can be done with a cell phone call or a text message). The patient may be automatically queried for his location, in the event he becomes disabled and intervention is needed.

Treatment options provided and displayed by the system menus (or by a health care professional) include:
Recommending administering or adjusting carbohydrate sources to respond to blood glucoses in specified ranges;
Recommending maintaining proper hydration;
Recommending collecting other data to assess status, such as urine ketones, last activity update, or symptoms of intercurrent illness;
Recommending adjustments in exercise or physical activity, or ceasing activity in appropriate cases;
Recommending adjustment or administration of medications including insulin; and
Recommending reducing stress or seeking treatment for infection or illness.

For a patient who has implemented the treatment prescribed by the system (i.e., by the menu or a health care professional), at subsequent intervals the effectiveness of such treatment is monitored, again, by transmitting the patient's blood glucose level and some of the additional parameters (exercise level, e.g., from the pedometer, diet, and possibly others) to the server. The server analyzes the results, and may select a decision support menu for the patient to gather additional clinically-relevant information, recommend modifications to the treatment, or initiate intervention and expert consultation. As the patient response database accumulates, the server is able to provide recommendations that are increasingly personalized and appropriate in view of that patient's historical responses.

In a preferred embodiment, the menu content is provided by the server, and not stored on the phone, or Bluetooth or other wireless device (XML format is preferred for the data). An integrated voice recognition system may also be provided—as this is convenient for cell phone/Bluetooth. Storing menu content on the server allows the menus to be readily updated, without any need for participants to download the updates. Constant updating of menus is anticipated, as research advances, and user experience is gained.

The system also allows users to remotely access from educational materials (videos, audios, text) relating to diabetes. In some cases the decision support system may direct the user to particular educational materials, for example, if the user appears to be repeatedly violating treatment principles.

The system also would track and compile each users' data, and allow provision of periodic reports, summaries, and longer term analysis.

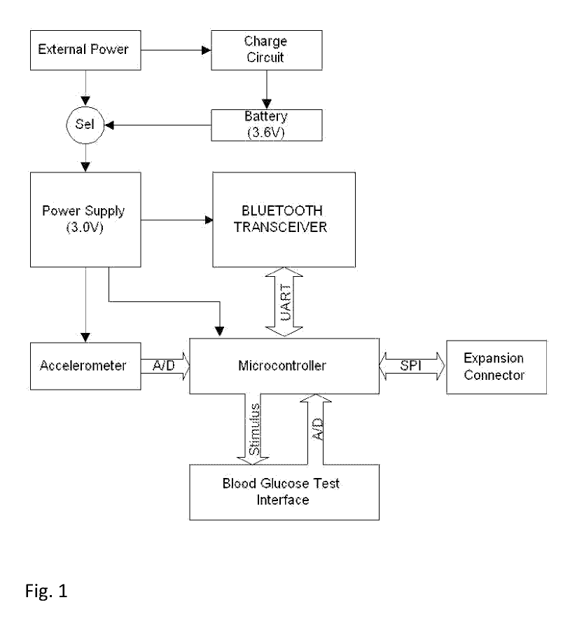

An embodiment of a wireless glucometer and pedometer is shown in FIG. 1, which represents the system design with a microcontroller to receive data, an accelerometer for the pedometer function, a Bluetooth receiver, and a blood glucose test interface, as well as power sources.

This embodiment in FIG. 1 could encompass the microcontroller along with the required electronics to support the reading of the glucometer test strips (shown as “blood glucose test interface” in FIG. 1), or, all of the items shown in FIG. 1.

The basic embodiment of a wireless glucometer and pedometer would include the microcontroller, interface electronics required to read the glucose test strips including the test strip socket, a TTL UART (Universal Asynchronous Receive Transmit port) interface and an SPI (Serial Peripheral Interface). The SPI and UART should be direct peripherals of the microcontroller and would be accessed via a micro connector. This design would be powered from 3.0 V DC and would not require a power supply in the design. The filtering required would be standard bypass capacitors as needed.

This supplemented embodiment would include everything shown in FIG. 1 and would build upon the components in the basic design. The supplemented embodiment includes two power sources, but could be designed, preferably, such that only one of the two power sources is utilized; i.e., preferably an external 3.6V power source, or an internal 3.6V battery with an associated charge circuit.

A microcontroller should be selected that can meet the minimum requirements to support the associated devices (e.g., glucometer and pedometer) and also support the software application, while remaining small in physical size. Salient features of the microcontroller include a UART, SPI, A/D (Analog to Digital Converter), and general I/O (Input/Output pins).

Beyond the A/D and general I/O pins required for reading the glucose test strips, two A/D inputs will also be required by the accelerometer, which also requires one UART and one SPI interface.) The purpose of the SPI interface is to allow devices to be added to the glucometer without a complete product redesign.

A command language will be utilized in the format of:

1 byte CMD, 1 byte Size of Data, Data bytes to follow, checksum, CR
The command format is as follows:

| | Number of | | |
|---|---|---|---|
| Command | Bytes to | Bytes to | |
| Byte: | Follow | Follow | Check Sum |
| A one byte | Number of | The actual | The additive single |
| command | bytes | bytes | byte result of the |
| | following | following | Command, Number of |
| | the command | the command | Bytes to Follow, |
| | | | and Bytes to Follow. |

These bidirectional commands would allow the server or monitoring station to gain access to data such as:

| | | |
|---|---|---|
| Glucose Reading | Command 7EH | Request a blood glucose reading |
| Error Messages | Command 90H | Get error messages |
| Send Calibration | Command 80H | Send blood glucose strip |
| Data | | calibration data |
| Accelerometer Data | Command 81H | Send accelerometer 3 axis data |
| Access to | Command 82H | Send data to the Bluetooth |
| Bluetooth Radio | | Radio |

The glucometer will have one button for power and other features (depending upon the length of the button press). The glucometer will also have a coded (blue) lead which is connected to the Bluetooth Radio, and a bi-color (e.g., red/green lead) for defined use.

The preferred embodiment also requires a Bluetooth radio, having as its required profile, an SPP (Serial Port Profile). The design of the Bluetooth radio must consider a multitude of factors, including RF characteristics, antenna, Bluetooth Stack, FCC approval, and Bluetooth certification. Bluetooth radio capability can be added with minimal effort by using the National Semiconductor LMX9838 (release date Sep. 15, 2007). The design issues are largely avoided because the LMX9838 is a “drop-in” Bluetooth solution. Many other Bluetooth integrated circuits are available and can also be used to provide the Bluetooth solution.

The glucometer will make all data available over the Bluetooth Radio. The main communication between the Bluetooth Radio and the microcontroller is through the UART port. The UART speed will be set to 9600 Baud initially, though speeds of up to 115K Baud can be used. The bidirectional commands in the tables above will be supported via the Bluetooth Radio using the SPP profile.

In the preferred embodiment, one variant will require external power from a 3.6V battery source. A male mini USB connector built onto the circuit board would connect directly to the 3.6V battery power source, which could be a rechargeable lithium ion cell or a nickel metal hydride cell. USB functionality is not required. A power supply would be required to regulate the 3.6V battery power down to the system 3.0V requirement.

A battery charge circuit will also be required. The source voltage for the charge circuit is 5 VDC. The circuit board should have a mini USB female connector. USB communication is not required in order to charge the battery. This eliminates the need to use a wall transformer, but does not prevent the use of a wall transformer.

In the preferred embodiment, a Freescale accelerometer MMA6270Q will optionally be added to the circuit board. Beyond the 3.0V power requirement, this feature will also require 3 I/O lines and 2 A/D lines from the microcontroller.

Beyond the functional requirements, the goal of the design is to produce a functioning unit as small as possible, preferably with outline dimension of about 45 mm×13 mm×13 mm, or, not more than 25% larger. The unit preferably has a plastic housing designed to include either a swivel or retractable guard and accommodates all the electronics packages for operation.

An example of an interaction between server and patient is as follows:

### Urgent Responses for High or Low Individual or Trending Values

Individual Blood Glucose (BG) reading response (single value response)

| BG | |
|---|---|
| Level | Response |
| <=40 | Receive a call from a call center triage person to |
| | check status. Message that says “Use caution. |
| | Your BG is very low. Eat a simple sugar snack (15 g carbs) |
| | and recheck BG in 15 minutes” |
| 41-70 | Message that says “Your BG is below the target range. |
| | Eat a simple sugar snack (15 g carbs) and recheck BG in |
| | 15 minutes.” |
| 241-400 | Message: “Your BG is high. Check ketones? Treat as |
| | per your physician's recommendations.” Unless |
| | Change from previous BG is >80 points above current reading |
| | (e.g. if prior reading was 400 and >1 hour ago, and current |
| | reading is 290, no further message). Then no message should |
| | be generated. |
| >400 | Receive a call from a call center triage person to check status. |
| | Message: “Use caution and treat as per your physician's |
| | recommendations.” |

Trending Values responses

| BG Level | Response |
|---|---|
| 3 Readings <60 in | Message that says you should seek advice from your |
| 24-48 hours | health care provider |
| 5 consecutive | Message: “You should seek advice from your health |
| readings >240 | care provider to address BG levels.” |
| 3 consecutive | Message: “You should seek advice from your health |
| readings >350 | care provider to address BG levels.” |

A system of reward points for users is also contemplated, for reporting of blood glucose by users, to encourage them to use the system and to keep their BG in a desired range. Reward points could also be utilized by payors (insurance providers) as further incentive for participation in preventive care. One embodiment of a system of reward points is as follows.
Basic 5 points for a blood glucose report
Additional reward points are given for the following conditions.
   5 points for User-set Min Value<=BG<=User-set Max Value
   1 point for User-set Max Value<BG<=200

The reward points are calculated for each blood glucose report in HPA and sent to the users' cellphones. Accumulated reward points are maintained at the server.

For a reported blood glucose, result interpretation is given on the following basis:
“Too Low—TREAT” for BG<60
“Below Target” for 60<=BG<User-set Min Value
“On Target” for User-set Min Value<=BG<=User-set Max Value
“Above Target” for User-set Max Value<BG<=200
“Too High” for BG>200
   The result interpretation is checked for each blood glucose report in HPA and sent to the user's cellphones with reward points to show them to users.

What is claimed is:

1. A method for control and treatment of diabetes comprising:
monitoring, by the patient, at intervals, of his/her blood glucose level and at least one additional biometric or health-related parameter;
transmitting, at intervals, the blood glucose level and said additional parameter(s) from the individual to a server through a portable device carried by the individual, wherein, based on said blood glucose level and the additional parameter(s), a particular menu of possible treatment options is selected from a set of such menus and transmitted from the server to the individual;
monitoring the effectiveness of the treatment selected by the individual from the work flow-decision support menu by transmitting the patient's blood glucose level and at least one additional parameter at subsequent intervals to the server;
analyzing the subsequently transmitted patient blood glucose level and at least one additional parameter; and
providing updated and menus of possible treatment options to the patient based on the analysis, wherein the analysis includes analyzing the effectiveness of the previously prescribed treatments.

2. The method of claim 1 wherein the additional parameter is one or more of: the quantity of energy expended by the patient over a particular time period; blood pressure; heart rate; blood oxygenation levels; and blood chemistry including cholesterol including blood pressure, blood oxygenation levels, pulse rate, or blood chemistry including cholesterol and blood ketone levels.

3. The method of claim 2 wherein the quantity of energy expended is measured by a pedometer which is part of the portable device.

4. The method of claim 3 wherein the pedometer measures acceleration, and derives distance traveled by the patient, and thus energy expended based on the patient's mass.

5. The method of claim 1 wherein the transmitting to the server is performed using a wireless interface.

6. The method of claim 5 wherein the wireless interface is a cellular phone.

7. The method of claim 1 wherein the server stores the data from the patient.

8. The method of claim 1 wherein the particular menu transmitted to the patient depends on the blood glucose level and the other biometric or health-related parameter(s).

9. The method of claim 8 wherein the particular menu transmitted to the patient further depends on the personalized stored data for the patient by the server, based on data input previously for said patient.

10. The method of claim 1 wherein when the menu is transmitted when the patient's blood glucose level is determined to be outside a specified range.

11. The method of claim 10 wherein the upper limit of the specified range is a blood glucose level of 250 mg/dl.

12. The method of claim 10 wherein the upper limit of the specified range is a blood glucose level of 300 mg/dl.

13. The method of claim 1 wherein when the menu transmitted is readable on a wireless receiver or a computer.

14. The method of claim 1 wherein when the menu is transmitted as a voice message.

15. The method of claim 2 further including transmitting a different menu if the patient monitoring indicates that the selected treatment is not as effective as expected.

16. The method of claim 1 wherein if the blood glucose level or one or more of the additional parameters is outside a specified range, a health professional is automatically alerted.

17. The method of claim 1 wherein the menu provides for the patient selecting one or more of the following treatment options: begin or cease exercise; drink water; administer insulin or other medication; and, reduce infection, illness or stress.

18. The method of claim 1 wherein the blood glucose level and said additional parameter(s) are transmitted to the server automatically.

19. The method of claim 1 wherein the patient initiates transmission of the blood glucose level and said additional parameter(s) to the server.

20. The method of claim 19 wherein the transmission is of a voice message and the menu is an integrated voice recognition system.

21. The method of claim 1 wherein the blood glucose level and said additional parameter(s) are transmitted to the server in xml format.

22. The method of claim 5 wherein wireless interface does not separately store or process the blood glucose level and said additional parameter(s) or the menu provided from the server.

23. The method of claim 5 wherein the blood glucose level is monitored by the patient using a glucose test strip which is inserted into a reader associated with the wireless interface.

24. The method of claim 1 further including providing the server with information on controlling diabetes which can be transmitted to the patient.

25. The method of claim 1 wherein the menu provides the patient the option of having certain information on controlling diabetes transmitted to him/her.

25. The method of claim 24 wherein the information is automatically transmitted to the patient if the patient's blood glucose level or one or more of the additional parameters is outside a specified range.
